(19) United States
(12) Patent Application Publication
(10) Pub. No.: US 2018/0055484 A1
(43) Pub. Date: Mar. 1, 2018

(54) ULTRASONIC PROBE AND ULTRASONIC IMAGING APPARATUS

(71) Applicant: SAMSUNG MEDISON CO., LTD., Hongcheon-gun (KR)

(72) Inventors: Jae-Yk KIM, Seongnam-si (KR); Dong Soo JUN, Seoul (KR); Dong Hyun KIM, Suwon-si (KR); In Seong SONG, Daegu (KR)

(21) Appl. No.: 15/441,768

(22) Filed: Feb. 24, 2017

(30) Foreign Application Priority Data
Aug. 26, 2016 (KR) 10-2016-0109207

(51) Int. Cl. A61B 8/00; A61B 8/08; A61B 8/14

(52) U.S. Cl. CPC A61B 8/4494; A61B 8/0866; A61B 8/14; A61B 8/465; A61B 8/4272; A61B 8/56; A61B 8/4444; A61B 8/467; A61B 8/54

(57) ABSTRACT

An ultrasonic probe and an ultrasonic imaging apparatus are disclosed. The ultrasonic probe includes a vibration generator for transferring sound beneficial to a fetus so as to transmit the sound signal to the fetus more efficiently than a conventional ultrasonic probe. An ultrasonic probe includes a transducer configured to transmit an ultrasonic signal to an object or receive an ultrasonic signal reflected from the object. The ultrasonic probe includes a frame and a vibration generator. The vibration generator is located in the frame, and generates vibration when the ultrasonic probe contacts the object in such a manner that the generated vibration is transferred to a fetus of the object.

### CROSS-REFERENCE TO RELATED APPLICATION

This application claims the benefit of Korean Patent Application No. 10-2016-0109207, filed on Aug. 26, 2016 in the Korean Intellectual Property Office, the disclosure of which is incorporated herein by reference.

### BACKGROUND

### 1. Field

Embodiments of the present disclosure relate to an ultrasonic probe for acquiring an ultrasonic signal, and an ultrasonic imaging apparatus for imaging the acquired ultrasonic signal.

### 2. Description of the Related Art

An ultrasonic imaging system is one of the important imaging systems applicable to various technical fields, and has non-invasive and non-destructive characteristics. Accordingly, the ultrasonic imaging apparatus has been widely utilized for various medical fields. The ultrasonic imaging system acquires diagnostic images from the interior of a target object using an ultrasonic probe, and displays 2D or 3D images through an ultrasonic diagnostic device.

Noise may unexpectedly occur in the diagnostic process based on the ultrasonic probe. Such noise may be harmful to a target object (specially, a fetus) to be diagnosed, such that many developers and companies are conducting intensive research into a method for reducing noise and transmitting a sound signal beneficial to the fetus.

### SUMMARY

Therefore, it is an aspect of the present disclosure to provide an ultrasonic probe including a vibration generator for transferring sound beneficial to a fetus so as to transmit the sound signal to the fetus more efficiently than a conventional ultrasonic probe, and an ultrasonic imaging apparatus.

In accordance with one aspect of the present disclosure, an ultrasonic probe includes a transducer configured to transmit an ultrasonic signal to an object or receive an ultrasonic signal reflected from the object. The ultrasonic probe includes a frame and a vibration generator. The vibration generator is located in the frame, and generates vibration when the ultrasonic probe contacts the object in such a manner that the generated vibration is transferred to a fetus of the object.

The vibration generator may generate vibration by vibrating the frame such that vibration is transferred to the fetus of the object.

The vibration generator may contact the frame.

The ultrasonic probe may further include: a lens located at one surface of the transducer. The vibration generator generates vibration by vibrating the lens such that vibration is transferred to the fetus of the object.

The vibration generator may vibrate the lens through the frame such that vibration is transferred to the fetus of the object.

The vibration generator may contact the lens.

The ultrasonic probe may further include: a cap configured to enclose the transducer. The vibration generator generates vibration by vibrating the cap through the frame such that vibration is transferred to the fetus of the object.

The ultrasonic probe may further include: a lens located at one side of the transducer. The vibration generator generates vibration by vibrating at least one of the cap and an acoustic coupler disposed between the transducer and the cap through the lens such that vibration is transferred to the fetus of the object.

The vibration generator may contact at least one of the cap and the frame.

The ultrasonic probe may further include: a communicator configured to transmit and receive sound data to and from an external part; and a controller configured to control the vibration generator so as to generate vibration on the basis of the sound data.

The ultrasonic probe may further include: a storage part configured to pre-store sound data therein; and a controller configured to control the vibration generator so as to generate vibration on the basis of the sound data pre-stored in the storage part.

In accordance with another aspect of the present disclosure, an ultrasonic imaging apparatus includes: an ultrasonic probe configured to include a transducer for transmitting an ultrasonic signal to an object or receiving an ultrasonic signal reflected from the object; an image processor configured to acquire an image on the basis of the reflected ultrasonic signal; and a display configured to display the image acquired by the image processor. The ultrasonic probe includes: a frame and a vibration generator. The vibration generator may be located in the frame, and may generate vibration when the ultrasonic probe contacts the object in such a manner that the generated vibration is transferred to a fetus of the object.

The ultrasonic probe may generate vibration by vibrating the frame such that vibration is transferred to the fetus of the object.

The ultrasonic probe may further include a lens located at one surface of the transducer. The ultrasonic probe may generate vibration by vibrating the lens such that vibration is transferred to the fetus of the object.

The ultrasonic probe may vibrate the lens through the frame such that vibration is transferred to the fetus of the object.

The ultrasonic probe may further include: a cap configured to enclose the transducer. The ultrasonic probe may generate vibration by vibrating the cap through the frame such that vibration is transferred to the fetus of the object.

The ultrasonic probe may further include a lens located at one side of the transducer. The ultrasonic probe may generate vibration by vibrating at least one of the cap and an acoustic coupler disposed between the transducer and the cap through the lens such that vibration is transferred to the fetus of the object.

The ultrasonic probe may further include: a communicator configured to transmit and receive sound data to and from an external part; and a controller configured to control the vibration generator so as to generate vibration on the basis of the sound data.

The ultrasonic probe may further include: a storage part configured to pre-store sound data therein; and a controller configured to control the vibration generator so as to generate vibration on the basis of the sound data pre-stored in the storage part.

Additional aspects of the invention will be set forth in part in the description which follows and, in part, will be obvious from the description, or may be learned by practice of the invention.

### BRIEF DESCRIPTION OF THE DRAWINGS

These and/or other aspects of the invention will become apparent and more readily appreciated from the following description of the embodiments, taken in conjunction with the accompanying drawings of which:

FIG. 1 is a perspective view illustrating an external appearance of an ultrasonic imaging apparatus according to an embodiment of the present disclosure.

FIGS. 2A to 2C illustrate external appearances of ultrasonic probes according to different embodiments, and are exploded views illustrating the ultrasonic probes according to different embodiments.

FIG. 3 is a conceptual diagram illustrating a method for transmitting a sound signal using the ultrasonic probe according to an embodiment of the present disclosure.

FIG. 4 is a control block diagram illustrating the ultrasonic probe according to an embodiment of the present disclosure.

FIGS. 5A and 5B are cross-sectional views illustrating the ultrasonic probe according to an embodiment of the present disclosure.

FIGS. 6A to 6C are a cross-sectional view and an exploded view illustrating the ultrasonic probe according to an embodiment of the present disclosure.

FIG. 7 is a conceptual diagram illustrating communication between the ultrasonic probe and the external device according to an embodiment of the present disclosure.

### DETAILED DESCRIPTION

Reference will now be made in detail to the embodiments of the present disclosure, examples of which are illustrated in the accompanying drawings, wherein like reference numerals refer to like elements throughout.

The terms used in the present application are merely used to describe specific embodiments and are not intended to limit the present invention. A singular expression may include a plural expression unless otherwise stated in the context. In the present application, the terms “including” or “having” are used to indicate that features, numbers, steps, operations, components, parts or combinations thereof described in the present specification are present and presence or addition of one or more other features, numbers, steps, operations, components, parts or combinations is not excluded.

In description of the present invention, the terms “first” and “second” may be used to describe various components, but the components are not limited by the terms. The terms may be used to distinguish one component from another component. For example, a first component may be called a second component and a second component may be called a first component without departing from the scope of the present invention. The term “and/or” may include a combination of a plurality of items or any one of a plurality of items.

Throughout the specification of the present disclosure, if it is assumed that a certain part includes a certain component, the term ‘comprising or including’ means that a corresponding component may further include other components unless a specific meaning opposed to the corresponding component is written. In addition, another term ‘. . . part’, “. . . unit’, ‘module’ or the like means a unit for processing at least one function or operation, and this unit may be implemented by hardware, software, or a combination thereof. As used in the specification and appended claims, the terms “a”, “an”, “one”, “the” and other similar terms include both singular and plural forms, unless context clearly dictates otherwise.

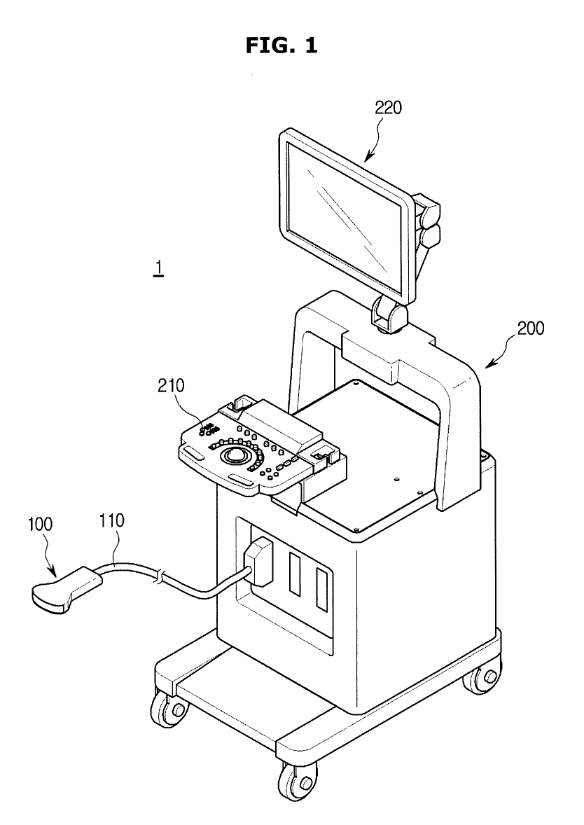

FIG. 1 is a perspective view illustrating an external appearance of an ultrasonic imaging apparatus according to an embodiment of the present disclosure.

Referring to FIG. 1, the ultrasonic imaging apparatus 1 may include an ultrasonic probe 100 and a main body 200. The ultrasonic probe 100 may transmit an ultrasonic signal to a target object, may receive an echo ultrasonic signal from the target object, and may convert the received echo ultrasonic signal into an electrical signal to obtain an ultrasonic image. The main body 200 may be connected to the ultrasonic probe through a cable 100, and may display an ultrasonic image.

The ultrasonic probe 100 may be connected to the main body 200 through the cable 100, may receive various signals needed to control the ultrasonic probe 100, or may transmit an analog or digital signal corresponding to the ultrasonic echo signal received by the ultrasonic probe 100. In addition, the ultrasonic probe 100 may transmit information regarding the operation state of the ultrasonic probe 100 to the main body 200.

The main body 200 may be used for ultrasonic diagnosis in hospitals or the like as shown in FIG. 1. However, the scope or spirit of the main body 200 is not limited to FIG. 1.

The main body 200 of FIG. 1 may include an input part 210 and a display 220. The input part 210 may receive setup information related to the ultrasonic probe 100, various control commands, etc. from the user.

In accordance with the embodiment, setting information of the ultrasonic probe 100 may include gain information, zoom information, focus information, time gain compensation (TGC) information, depth information, frequency information, power information, frame average information, dynamic range information, etc. However, the scope or spirit of the setup information of the ultrasonic probe 100 is not limited thereto, and the setup information may include various kinds of information capable of being established for ultrasonic imaging.

In accordance with another embodiment, the input part 210 may receive a control command related to the ultrasonic probe 100 or the main body 200 from the user. The above-mentioned information or command may be transmitted to the ultrasonic probe 100 through the cable 110, and the ultrasonic probe 100 may be established according to the received information.

The input part 210 may be implemented as a keyboard, a foot switch or a foot pedal. For example, the keyboard may be implemented by hardware. The keyboard may include a switch, a key, a joystick, a track ball, etc. In another example, the keyboard may also be implemented by software such as a graphical user interface (GUI). In this case, the keyboard may be displayed on the display 220. The foot switch or the foot pedal may be located below the main body 200, and the user may control the main body 200 using the foot pedal.

The display 220 may display an ultrasonic image regarding a target site contained in the target object. The ultrasonic image displayed on the display 220 may be a 2D ultrasonic image or a 3D ultrasonic image. Various ultrasonic images may be displayed according to the operation mode of the ultrasonic imaging apparatus. In addition, the display 220 may display menus or information related to the ultrasonic image, and may display information regarding the operation state of the ultrasonic probe 100.

The display 220 may be implemented by any well-known display panel, for example, a Cathode Ray Tube (CRT), a Liquid Crystal Display (LCD), a Light Emitting Diode (LED), a Plasma Display Panel (PDP), an Organic Light Emitting Diode (OLED), etc., without being limited thereto.

If the display 220 is implemented as a touchscreen, the display 220 may also act as the input part 210. That is, the user may input various commands through the display 220 or the input part 210.

Meanwhile, the ultrasonic imaging apparatus 1 may include an image processor configured to convert the received echo ultrasonic signal into an ultrasonic image. The image processor may be implemented by hardware such as a microprocessor, or may be implemented by software capable of being executed in hardware.

The image processor may acquire image information through image processing of the ultrasonic image. In this case, the image information may be information regarding images contained in the respective pixels contained in the ultrasonic image, and may include brightness, color, illumination, chromatic contrast, etc. of the respective pixels.

The ultrasonic probe 100 according to the embodiment of the present disclosure will hereinafter be described with reference to the attached drawings.

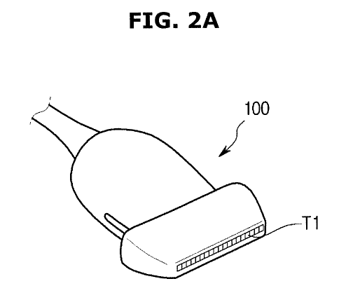

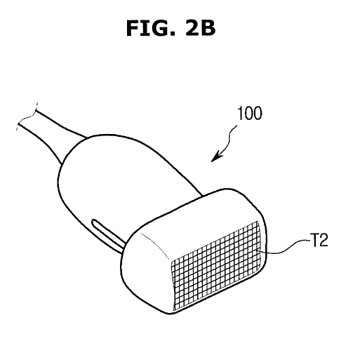

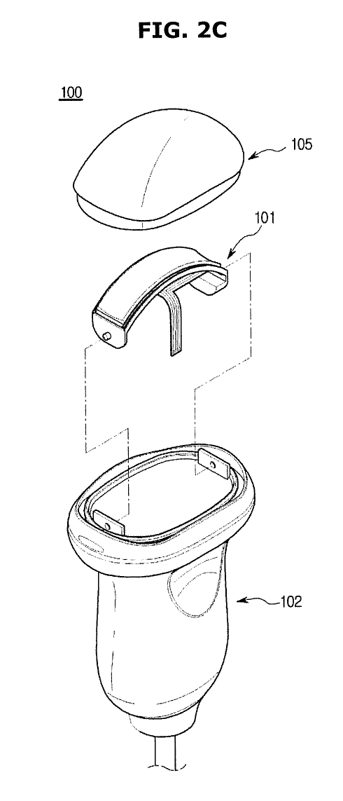

FIGS. 2A to 2C illustrate external appearances of ultrasonic probes according to different embodiments, and are exploded views illustrating the ultrasonic probes according to different embodiments.

Referring to FIGS. 2A to 2C, the ultrasonic probe 100 may contact the surface of the target object, and may transmit the ultrasonic signal to the target object. In more detail, the ultrasonic probe 100 may transmit the ultrasonic signal to the interior of the target object upon receiving a control command signal from the main body 200, may transmit the ultrasonic signal to the interior of the target object, may receive the echo ultrasonic signal reflected from a specific site contained in the target object, and may transmit the received echo ultrasonic signal to the main body 200. Therefore, the ultrasonic probe 100 may transmit the echo ultrasonic signal received from the target object to the main body 200, or may acquire the ultrasonic image from the echo ultrasonic signal and transmit the acquired ultrasonic image. However, the scope or spirit of the ultrasonic probe 100 is not limited thereto.

The transducer 101 may include a piezoelectric layer for converting an electrical signal into a sound signal and vice versa through vibration of a piezoelectric material; a sound matching layer for reducing a difference in acoustic impedance between the piezoelectric layer and the target object so as to maximally transmit an ultrasonic signal generated by the piezoelectric layer to the target object; a lens layer for focusing the ultrasonic signal proceeding in a forward direction of the piezoelectric layer to a specific position; and a backing layer (also called a sound absorption layer) for preventing image distortion by preventing the ultrasonic signal from proceeding in a backward direction of the piezoelectric layer.

In this case, the ultrasonic probe 100 may include a transducer array for converting an electrical signal into an ultrasonic signal and vice versa to transmit ultrasonic signals to the interior of the target object. The transducer array may include a plurality of transducer elements.

The transducer 101 may include the transducer array, and the transducer array may include a plurality of transducer elements. The transducer array may be a one-dimensional (1D) array or a two-dimensional (2D) array. For example, as can be seen from FIG. 2A, the transducer module may include a one-dimensional (1D) array transducer T1. In another example, as can be seen from FIG. 2B, the transducer 101 may include a 2D array transducer T2.

For example, respective transducer elements constructing the 1D transducer array may convert ultrasonic signals into electrical signals and vice versa. For this purpose, each of the transducer elements may be implemented by any of a magnetostrictive ultrasonic transducer using magnetostrictive effects of a magnetic material, and a piezoelectric ultrasonic transducer or piezoelectric micromachined ultrasonic transducer (pMUT) using the piezoelectric effects of a piezoelectric material. If necessary, the transducer element may also be implemented as a capacitive micromachined ultrasonic transducer (cMUT) to transmit and receive ultrasonic waves using vibration of several hundred or thousands of micromachined thin films.

Referring to FIG. 2B, the transducer 101 may also include the 2D array transducer T2. If the transducer 101 includes the 2D array transducer T2, the transducer 101 may perform 3D imaging of the interior of the target object.

The respective transducer elements constructing the 2D array transducer are identical to those of the 1D array transducer, and as such a detailed description thereof will herein be omitted for convenience of description and better understanding of the present disclosure.

Referring to FIG. 2C, the ultrasonic probe 100 may include a rotatable transducer 101. The ultrasonic probe 100 may include a frame 102 grasped by a user who desires to use the transducer 101. A cap 105 contacting the target object to be diagnosed may be arranged at the front end of the frame 102. The transducer 101 may be contained in the cap 105.

The transducer 101 may include an ultrasonic vibrator to transmit and receive ultrasonic signals. The transducer 101 may be rotatably installed in the cap 105 so that the transducer 101 can read the 3D image of the target object to be diagnosed.

The transducer 101 may be mounted to a rotational shaft. The rotational shaft may receive driving power from the driving device so that the rotational shaft can rotate. If the rotational shaft rotates, the transducer 101 mounted to the rotational shaft can also rotate.

The cap 105 may include an inner surface facing an outer surface of the transducer 101 in a manner that a constant spacing between the inner surface of the cap 105 and the outer surface of the transducer 101 can be maintained during rotation of the transducer 101. For example, the outer surface of the transducer 101 and the inner surface of the cap 105 may be formed in an arc shape having the same central point.

The inner space of the cap 105 may be filled with an acoustic coupler acting as a medium through which ultrasonic signals generated by the transducer 101 can be transferred.

The ultrasonic probe 100 may include a vibration generator 103 therein, and a detailed description thereof will hereinafter be given.

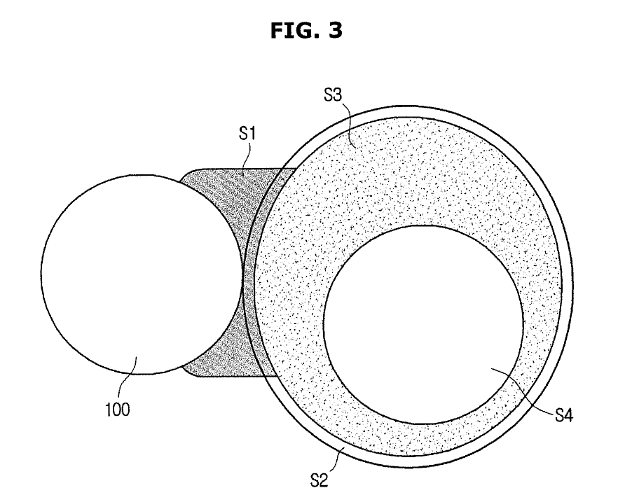

FIG. 3 is a conceptual diagram illustrating a method for transmitting a sound signal using the ultrasonic probe 100 according to an embodiment of the present disclosure.

According to an audible route of a user, eardrums of the user vibrate due to air vibrations so as to vibrate lymph fluid in the cochlea, such that the user can listen to various sound signals through the auditory nerves.

However, according to bone conduction vibration used by a vibration generator of the embodiment, sound waves vibrate the skull or cranium of the user so that the resultant sound waves arrive at the inner ears and finally arrive at the auditory nerves. Bone conduction may be classified into indirect bone conduction in which external sound waves arrive at the inner ears after passing through the skull at loud sound of 2 kHz or higher; and direct bone conduction in which vibration of an object contacting the head or other vibration generated in the human body arrives at eardrums or middle ears and finally arrives at the inner ears. The vibration generator 103 operated by bone conduction does not receive the external sound using vibration transmission, so that the vibration generator 103 can effectively transfer sound waves to the fetus.

Referring to FIG. 3, the ultrasonic probe 100 may generate the sound signal by the vibration generator 103 mounted to one surface of the ultrasonic probe 100. The transmission position of the sound signal of the ultrasonic probe 100 will hereinafter be described in detail. The sound signal (i.e., vibration) generated by the ultrasonic probe 100 may be transferred to a skin S2 of the target object after passing through a gel S1.

The ultrasonic probe 100 may operate in close contact with the skin S2 of the target object through the gel S2, such that it can easily transfer vibration generated by the ultrasonic probe 100. The sound signal transferred to the skin S2 of the target object may be transferred to the amniotic fluid S3 enclosing the fetus S4. The amniotic fluid S3 may act as a medium through which the sound signal received from the skin S3 of the target object can pass. The sound signal may be transferred to bones of the fetus after passing through the amniotic fluid S3, and then be transferred to the auditory nerves of the fetus. Through the above-mentioned scheme, the sound signals are not transferred to the outside, and the sound signals can be transferred by contacting the skin of the target object. The ultrasonic probe 100 for use in the above-mentioned vibration transmission process will hereinafter be described in detail.

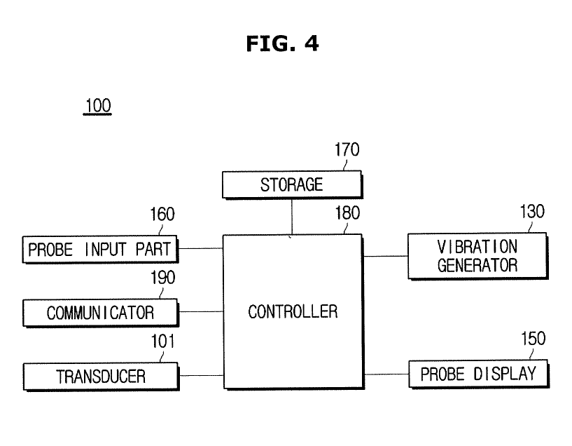

FIG. 4 is a control block diagram illustrating the ultrasonic probe 100 according to an embodiment of the present disclosure.

Referring to FIG. 4, the ultrasonic probe 100 may include a probe display 150, a probe input part 160, and a controller 180. The ultrasonic probe 100 may further include a transducer 101.

The transducer 101 may transmit ultrasonic signals to the target object, and may receive the echo ultrasonic signal reflected from the target object.

The transducer 101 may acquire ultrasonic images from the echo ultrasonic signal. In addition, assuming that the ultrasonic probe 100 includes the 2D array transducer or the 1D array transducer moving in the elevation direction, the transducer 101 may also generate 3D ultrasonic images using a plurality of 2D cross-sectional images.

The controller 180 may be integrated in a System on Chip (SoC) embedded in the ultrasonic probe 100. In this case, only one SoC may not be included in the ultrasonic probe 100, such that the scope or spirit of the present disclosure is not limited to only one SoC.

The controller 180 may generate vibrations by controlling the vibration generator 103, and may control the vibration generator 103 upon receiving sound data (i.e., sound source data) from a communicator 190.

A storage part 170 may store various kinds of data therein. The storage part may store data regarding the ultrasonic image acquired from the transducer 101, image information acquired from the ultrasonic image, etc.

In addition, the storage part 170 may store data regarding air images per mode. Here, the air images may denote ultrasonic images obtained when the ultrasonic signal is reflected from the air. The storage part 170 may store sound data used as a basis of vibrations to be generated by the vibration generator 103. For example, the sound data (sound source data) may include MP3, MPEG2 BC, MPEG2 AAC, RealAudio G2, LiquidAudio, WMA, and MP3 Pro.

The storage part 170 may be implemented as at least one of a flash memory type, a hard disk type, a multimedia card micro card, a card type memory (e.g. a Secure Digital (SD) memory or an eXtreme Digital (XD) memory), a Random Access Memory (RAM), a Static Random Access Memory (SRAM)), a Read Only Memory (ROM), an Electrically Erasable Programmable Read Only Memory (EEPROM), a Programmable Read Only Memory (PROM), a magnetic memory, a magnetic disk, an optical disk, etc., without being limited thereto.

The communicator 190 may transmit and receive various kinds of data to and from the main body 200 through the cable. The communicator 190 may transmit and receive various kinds of data (e.g., ultrasonic images, echo ultrasonic signals, Doppler data, etc.) related to diagnosis of the target object to and from the main body 200 through the cable. In addition, the communicator 190 may receive various kinds of control command signals from the main body 200.

The communicator 190 may include one or more constituent elements capable of wirelessly communicating with the external device, and may receive sound data from the external device, and as such a detailed description thereof will hereinafter be given.

Meanwhile, the ultrasonic probe 100 may include a probe display 150 therein. The probe display 150 may be implemented by any of well-known display panels, for example, CRT, LCD, LED, PDP, OLED, etc. without being limited thereto. The probe display 150 may display information related to the operation state of the ultrasonic probe 100, for example, power-supply state information of the ultrasonic probe 100. Specifically, the probe display 150 may display information related to the sound data used as a basis of vibration generated by the vibration generator 130.

In addition, the ultrasonic probe 100 may include a probe input part 160. The probe input part 160 may be implemented as a switch, a key, etc., without being limited thereto. The probe input part 160 may receive power ON/OFF commands of the ultrasonic probe 100 from the user, and may receive a control command related to vibration generated by the vibration generator 103. For example, the probe input part 160 may receive various control commands, for example, the type of sound source, the magnitude of sound source, the effect of vibration, etc. The probe input part 160 may also receive a control command related to the operation mode change of the ultrasonic probe 100, without being limited thereto.

The ultrasonic probe 100 may include the vibration generator 103. The vibration generator 103 may be implemented as a bone conduction speaker. A general speaker may be a device for generating audible sound waves by applying an alternating current (AC) having an audible frequency, and may allow an operation part located in a magnetic field region to vibrate due to AC (Alternating Current) magnetic flux generated by a negative current flowing in the coils. Although a general speaker may include a coil, a magnet, a vibration plate, and a diaphragm for maximizing vibration of the vibration plate, vibration generated by the vibration generator 103 according to the embodiment may be directly applied to the human body, and may stimulate the auditory nerves after passing through the skull, such that the speaker may be implemented as a bone conduction speaker including no diaphragm.

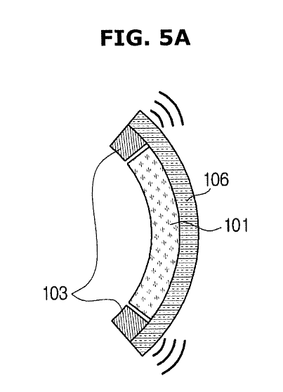

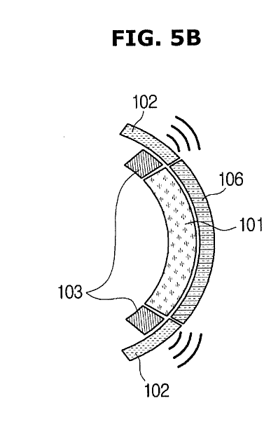

FIGS. 5A and 5B are cross-sectional views illustrating the ultrasonic probe 100 according to an embodiment of the present disclosure.

FIGS. 5A and 5B are cross-sectional views illustrating the ultrasonic probe 100 shown in FIG. 2.

Referring to FIG. 5A, the vibration generator 103 may be provided at one side of the transducer 101. In addition, the ultrasonic probe 100 may further include a lens 106 at one side of the transducer 101. Ultrasonic waves generated by the transducer 101 may be focused onto the lens 106. The lens 106 may be formed of silicon, rubber, etc. having acoustic impedance similar to acoustic impedance of the target object. The lens 106 may be formed in a convex type, the center part of which has a convex surface, or may be formed in a linear type, the center part of which has a flat surface. The vibration generator 103 may be located at one surface of the lens 106. As described above, the vibration generator 103 may output the sound signal received through bones of the target object to be diagnosed, such that the sound signal may not spread out to the outside. The lens 106 may contact the skin of the target object, and may transmit vibration, so that the lens 106 may act as the medium of vibration. The vibration generator 103 shown in FIG. 5A may be mounted to one surface of the lens 106 so that it can transmit the generated vibration to the lens 106. During the diagnostic process using the ultrasonic probe 100, the lens 106 must contact the target object, so that the vibration generator 103 can easily transmit vibration to the target object. Although the vibration generator 103 is located not only at both sides of the transducer 101 but also at the bottom surface of the lens 106 contacting the target object as shown in FIG. 5A, assuming that only one surface of the ultrasonic probe 101 capable of vibrating the lens 106 is used, the position of the vibration generator 103 is not limited thereto.

Referring to FIG. 5B, the vibration generator 103 may be provided at one surface of the frame 102 of the ultrasonic probe 101. The ultrasonic probe 100 may include the frame 102, and the frame 102 may include the vibration generator 103 and the transducer 101. The vibration generator 103 may be provided at one surface of the frame 102 so as to vibrate the frame 102. During ultrasonic diagnosis, the frame 102 contacts the skin of the target object, such that signals generated by the vibration generator 103 may be transferred to the frame 102. Therefore, although the vibration generator 103 shown in FIG. 5B is located not only at both sides of the transducer 101 but also at the bottom surface of the frame 102 contacting the target object, assuming that only one surface of the ultrasonic probe 101 capable of vibrating the frame 102 is used, the position of the vibration generator 103 is not limited thereto.

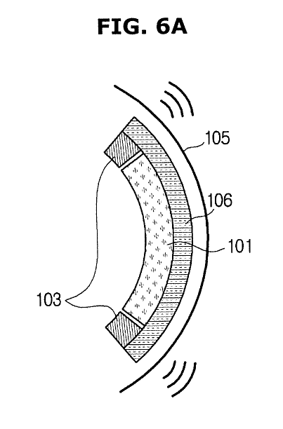

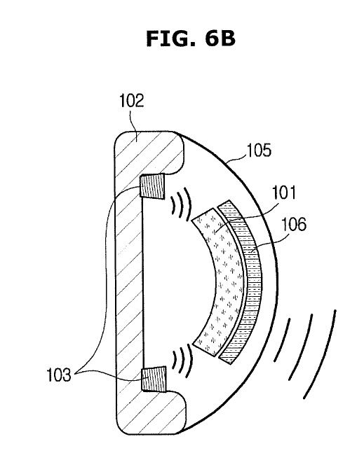

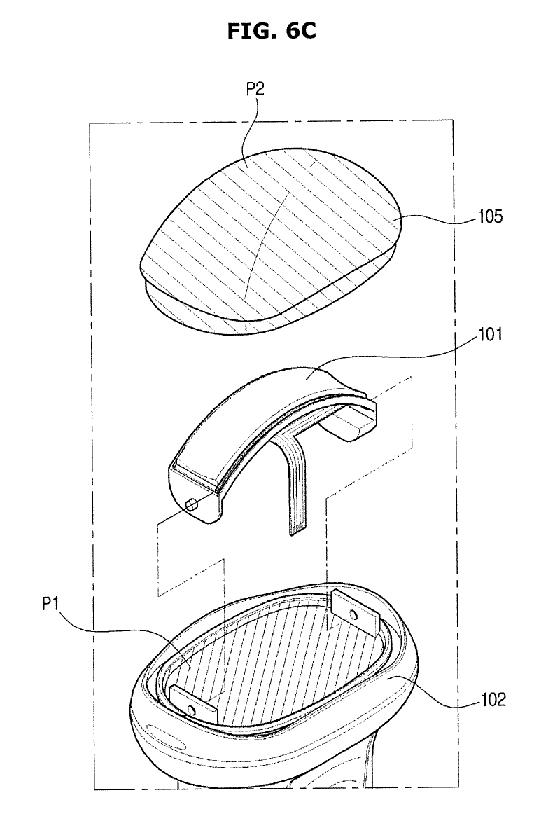

FIGS. 6A to 6C are a cross-sectional view and an exploded view illustrating the ultrasonic probe 100 shown in FIG. 2C according to an embodiment of the present disclosure.

The ultrasonic probe 100 shown in FIG. 6 may be a probe to acquire 3D images, and the transducer 101 of the ultrasonic probe 100 may operate or move. As described above, the ultrasonic probe 100 may include the cap so that a constant spacing between the ultrasonic probe 100 and the transducer 101 may be maintained, and a coupler may be disposed in the spacing between the transducer 101 and the cap 105.

Referring to FIG. 6A, the transducer 101 may include the lens 106 in the same manner as in FIG. 5. Therefore, the ultrasonic probe 101 of FIG. 6 may include the vibration generator 103 to vibrate the lens 106. The vibration generator 103 may vibrate the lens 106, and may also vibrate the coupler contacting the lens 106. If the coupler vibrates, the cap 105 of the ultrasonic probe 101 contacting the target object may also vibrate. In brief, the ultrasonic probe 100 of FIG. 6 may allow vibration generated by the vibration generator 103 to be transferred to the cap 105 through the lens 106 and the coupler.

FIG. 6B is a view illustrating the vibration generator 103 mounted to one surface of the frame 102.

FIG. 6C is an exploded view illustrating the ultrasonic probe 100 of FIG. 6B.

Referring to FIGS. 6B and 6C, the ultrasonic probe 100 may include the vibration generator 103 located at one surface of the frame 102 so that the frame 102 can vibrate. Since the frame 102 may directly contact the cap 105, vibration can be directly transferred to the cap 105 and finally arrives at the coupler. If vibration arrives at the coupler, the oil vibrates the cap 105 so that the sound signal can arrive at the target object. The ultrasonic probe 100 of FIG. 6B includes the spacing between the transducer 101 and the frame 102 in a different way from the ultrasonic probe 100 of FIG. 5B, and the vibration generator 103 may be located at many more installation positions than the ultrasonic probe 100 of FIG. 5B.

Referring to FIG. 6C, the vibration generator 103 may be located at the positions (P1, P2) of the frame 102 and the cap 105 of the ultrasonic probe 100, respectively. Sound signals can be transmitted to the fetus according to the above-mentioned scheme because the frame 102 or the cap 105 of the ultrasonic probe 100 vibrates, such that the vibration generator 103 may be located anywhere that the vibration generator 103 can vibrate within the frame 102. In conclusion, assuming that the vibration generator 103 is mounted to one surface of the frame and vibrates any one of the acoustic coupler, the cap 105, and the frame 102, the sound signal may finally arrive at the fetus. The vibration generator 103 may be located at one surface P2 of the cap 105 shown in FIG. 6C. In this case, the vibration generator 103 may directly transmit vibration to the cap 105. The vibration generator 105 may be mounted anywhere that the vibration generator 105 can transmit the sound signal by vibrating the frame 102 and the cap 105.

However, the installation positions of the vibration generators 103 shown in FIGS. 5 and 6 may not be limited thereto, and the vibration generator 103 may be mounted anywhere that the vibration generator 103 can vibrate any one of the lens 106, the frame 102, the cap 105, and the coil. Although two vibration generators 103 are exemplarily shown in FIGS. 5 to 6 for convenience of description, the number of the vibration generators 103 may be at least one as necessary.

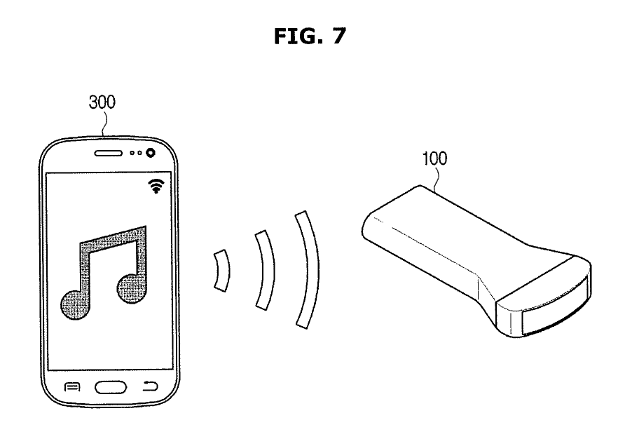

FIG. 7 is a conceptual diagram illustrating communication between the ultrasonic probe 100 and the external device 300. Referring to FIG. 7, the communicator 190 of the ultrasonic probe 100 may wirelessly communicate with the external device 300 through at least one of a near field communication (NFC) module and a mobile communication module.

The external device 300 may be implemented as any one of a laptop, a desktop computer, and a tablet PC, or may be implemented as a smartphone as shown in FIG. 8. The external device may include a mobile terminal such as a PDA, a watch detachably coupled to the user's body, and a glasses-type wearable terminal.

However, the external device 300 is not limited thereto, and may include any device which includes the communicator therein so as to communicate with the ultrasonic probe 100 over the wireless communication network and can transmit sound sources without departing from the scope or spirit of the present disclosure.

The ultrasonic probe 100 may communicate with the external device 300 using a communication module. The communication module may include one or more constituent elements capable of communicating with the external device 300. For example, the communication module may include Wireless LAN, Wi-Fi, Bluetooth, ZigBee, Wi-Fi Direct (WFD), Ultra wideband (UWB), Infrared Data Association (IrDA), Bluetooth Low Energy (BLE), Near Field Communication (NFC), etc. without being limited thereto.

The external device 300 may transmit and receive RF signals to and from at least one of a base station (BS), an external terminal, and a server over the mobile communication network. For example, the mobile communication module may transmit and receive various types of data to and from the external device after passing through the BS over the 3G or 4G communication network.

The external device 300 may receive sound data through a communication network, and may transmit the received sound data to the ultrasonic probe 100. The sound data may be digitized, and may include MP3, MPEG2 BC, MPEG2 AAC, RealAudio G2, Liquid Audio, WMA, and MP3 Pro.

As is apparent from the above description, according to the ultrasonic probe and the ultrasonic imaging apparatus, the ultrasonic probe according to the embodiment of the present disclosure includes a vibration generator for transferring sound beneficial to a fetus, such that the ultrasonic probe can transmit the sound signal to the fetus more efficiently than a conventional ultrasonic probe.

Although the above-mentioned embodiments of the present disclosure have been disclosed herein merely for illustrative purposes, the scope or spirit of the embodiments is not limited thereto, and those skilled in the art will appreciate that various modifications, additions and substitutions are possible, without departing from the scope and spirit of the disclosure as disclosed in the accompanying claims. For example, adequate effects of the present disclosure may be achieved even if the foregoing processes and methods may be carried out in different order than described above, and/or the aforementioned elements, such as systems, structures, devices, or circuits, may be combined or coupled in different forms and modes than described above or be substituted or switched with other components or equivalents.

Although a few embodiments of the present disclosure have been shown and described, it would be appreciated by those skilled in the art that changes may be made in these embodiments without departing from the principles and spirit of the invention, the scope of which is defined in the claims and their equivalents.

1. An ultrasonic probe including a transducer configured to transmit an ultrasonic signal to an object or receive an ultrasonic signal reflected from the object, comprising:
a frame; and
a vibration generator located in the frame, and configured to generate vibration when the ultrasonic probe contacts the object in such a manner that the generated vibration is transferred to a fetus of the object.

2. The ultrasonic probe according to claim 1, wherein the vibration generator generates vibration by vibrating the frame such that vibration is transferred to the fetus of the object.

3. The ultrasonic probe according to claim 2, wherein the vibration generator contacts the frame.

4. The ultrasonic probe according to claim 1, further comprising:
a lens located at one surface of the transducer,
wherein the vibration generator generates vibration by vibrating the lens such that vibration is transferred to the fetus of the object.

5. The ultrasonic probe according to claim 4, wherein the vibration generator vibrates the lens through the frame such that vibration is transferred to the fetus of the object.

6. The ultrasonic probe according to claim 4, wherein the vibration generator contacts the lens.

7. The ultrasonic probe according to claim 1, further comprising:
a cap configured to enclose the transducer, and
wherein the vibration generator generates vibration by vibrating the cap through the frame such that vibration is transferred to the fetus of the object.

8. The ultrasonic probe according to claim 7, further comprising:
a lens located at one side of the transducer, and
wherein the vibration generator generates vibration by vibrating at least one of the cap and an acoustic coupler disposed between the transducer and the cap through the lens such that vibration is transferred to the fetus of the object.

9. The ultrasonic probe according to claim 7, wherein the vibration generator contacts at least one of the cap and the frame.

10. The ultrasonic probe according to claim 1, further comprising:
a communicator configured to transmit and receive sound data to and from an external part; and
a controller configured to control the vibration generator so as to generate vibration on the basis of the sound data.

11. The ultrasonic probe according to claim 1, further comprising:
a storage part configured to pre-store sound data therein; and
a controller configured to control the vibration generator so as to generate vibration on the basis of the sound data pre-stored in the storage part.

12. An ultrasonic imaging apparatus comprising:
an ultrasonic probe configured to include a transducer for transmitting an ultrasonic signal to an object or receiving an ultrasonic signal reflected from the object;
an image processor configured to acquire an image on the basis of the reflected ultrasonic signal; and
a display configured to display the image acquired by the image processor,
wherein the ultrasonic probe includes:
a frame; and
a vibration generator located in the frame, and configured to generate vibration when the ultrasonic probe contacts the object in such a manner that the generated vibration is transferred to a fetus of the object.

13. The ultrasonic imaging apparatus according to claim 12, wherein the ultrasonic probe generates vibration by vibrating the frame such that vibration is transferred to the fetus of the object.

14. The ultrasonic imaging apparatus according to claim 12, wherein the ultrasonic probe further includes:
a lens located at one surface of the transducer,
wherein the ultrasonic probe generates vibration by vibrating the lens such that vibration is transferred to the fetus of the object.

15. The ultrasonic imaging apparatus according to claim 12, wherein the ultrasonic probe vibrates the lens through the frame such that vibration is transferred to the fetus of the object.

16. The ultrasonic imaging apparatus according to claim 12, wherein the ultrasonic probe further includes:
a cap configured to enclose the transducer, and
wherein the ultrasonic probe generates vibration by vibrating the cap through the frame such that vibration is transferred to the fetus of the object.

17. The ultrasonic imaging apparatus according to claim 15, wherein the ultrasonic probe further includes:
a lens located at one side of the transducer, and
wherein the ultrasonic probe generates vibration by vibrating at least one of the cap and an acoustic coupler disposed between the transducer and the cap through the lens such that vibration is transferred to the fetus of the object.

18. The ultrasonic imaging apparatus according to claim 12, wherein the ultrasonic probe further includes:
a communicator configured to transmit and receive sound data to and from an external part; and
a controller configured to control the vibration generator so as to generate vibration on the basis of the sound data.

19. The ultrasonic imaging apparatus according to claim 12, wherein the ultrasonic probe further includes:
a storage part configured to pre-store sound data therein; and
a controller configured to control the vibration generator so as to generate vibration on the basis of the sound data pre-stored in the storage part.
